(19) United States
(12) Patent Application Publication
(10) Pub. No.: US 2022/0346850 A1
(43) Pub. Date: Nov. 3, 2022

(54) MEDIAL TWIST ANCHOR

(71) Applicant: TIGON MEDICAL, Millersville, MD (US)

(72) Inventors: Jeremy Clark, Millersville, MD (US); Fotios Tjoumakaris, Millersville, MD (US); Umasuthan SRIKUMARAN, Ellicott City, MD (US)

(21) Appl. No.: 17/845,730

(22) Filed: Jun. 21, 2022

Related U.S. Application Data
(60) Provisional application No. 63/181,904, filed on Apr. 29, 2021.

(51) Int. Cl. A61B 17/86; A61F 2/08

(52) U.S. Cl. CPC A61B 17/8645; A61F 2/0811; A61B 17/8625; A61F 2002/0817; A61B 2017/044

(57) ABSTRACT

Disclosed are devices, methods and/or systems for securing tissue to bone, including a suture anchor implant having a body with an internal cavity and one or more helical cutting flutes positioned on an external surface of the body which direct at least a portion of the cut bony material into the internal cavity as the body is rotated into bone.

### CROSS-REFERENCE TO RELATED APPLICATIONS

This application claims the benefit of U.S. Provisional Patent Application Ser. No. 63/181,904 entitled “MEDIAL TWIST ANCHOR,” filed Apr. 29, 2021, the disclosure of which is incorporated by reference herein in its entirety.

### TECHNICAL FIELD

The invention relates to improved orthopedic tools and methods for use during orthopedic surgical procedures, including joint repair and/or replacement procedures. More specifically, disclosed are improved methods, apparatus and/or systems for securing and/or anchoring tissue structures to bones with a durable securement during surgery, including orthopedic repair of rotator cuff injuries.

### BACKGROUND OF THE INVENTION

There are a wide variety of suture anchor designs, related devices and/or surgical techniques for securing sutures, which can be passed through soft tissue, to bone. Many of such designs rely on the interference between external features of the anchor (i.e., barbs, ribs, ridges, etc.) and the bony inner surface of the fixation hole to provide fixation strength. Where there is a high amount of interference required, this may require a tighter fit between the anchor and the hole, resulting in a large force required to insert the anchor into the bone—which can result in broken anchors, broken insertion tools, damage to the suture and/or damage to the bone itself.

Many existing anchors and associated deployment tools incorporate multiple moving and interacting parts that require very tight tolerances in component manufacturing, which can significantly increase the expense and difficulty of device manufacture and assembly, and often negatively impact reliability of the anchor and related delivery systems. Moreover, many existing anchor designs do not facilitate harvesting and/or “packing” of bone graft within the anchor body—such actions may not even be possible with many designs, and with other designs the use of bone graft may require an additional surgical tools and/or additional assistance from another surgeon or assistant.

In addition, many current suture anchors used in medical procedures, such as rotator cuff repair, do not allow for more than one suture tape to be attached in a single anchor. A suture tape is a large flat suture which is used to hold the rotator cuff down to the bone for healing and is typically larger in size than a suture. A suture tape may be larger than a suture, and thus, due to its size, many current anchor designs may not be capable of supporting more than one of the larger suture tapes. Current anchors may be provided as cannulated (hollow) anchors or solid anchors. Solid anchors that may be strong enough to insert directly into the bone typically do not allow for the flow of biological healing products, such as bone marrow elements, from accessing the repair site. Thus, a need exists for a cannulated anchor which may support two or more suture tapes, may be inserted into a patient's bone in a single step, and which allows packing and/or venting of biologic elements directly into the anchor to promote healing and interdigitation.

### BRIEF SUMMARY OF THE INVENTION

The various inventions disclosed herein include the realization of a need for an improved suture anchor, system components and deployment tools, and related surgical methods for placing one or more anchoring sutures within a bone or other tissue structure. More specifically, the various suture anchors disclosed herein provide durable suture fixation and also provide for graft harvesting and use within the anchor during anchor placement and deployment.

In various embodiments, an anchor implant and associated system components are described which incorporate a bone harvesting system that draws cut biomaterial into the anchor body during anchor insertion—thereby packing the anchor with biomaterial which promotes bony ingrowth and long-term anchor fixation. The suture anchor may include an externally threaded portion, the threaded portion including: a body, one or more notched or cutaway flute surfaces, and one or more openings in a side of the body, the flute surface and the one or more openings configured to allow packing or venting of biologic elements directly into interior portions of the anchor.

In various embodiments, a reusable inserter shaft and other components can be utilized, which optionally greatly reduces waste and disposal of surgical tools and related components.

The present disclosure describes a suture or tissue anchor and associated deployment tools that are intended to secure suture or tissue to bone. There are many soft tissue to bone repair procedures, such as rotator cuff, SLAP (Superior Labral tear from Anterior to Posterior), and Bankart lesion repairs, or reconstruction of labral tissue to the glenoid rim, in which a surgeon needs to secure tissue in close contact with bone. Often the bone surface is roughened, and when tissue is pulled into intimate contact, the body's healing response will fuse the tissue and bone together. This suture is then passed through the soft tissue at the desired location, and the suture is secured to the anchor by tying a knot. Other methods include passing suture through the tissue first and then fastening the anchor and suture to the bone without knots.

In various aspects of the invention, there are disclosed methods for securing soft tissue to bone, which can comprise steps of driving an implantable anchor having a body distally into a desired bone site using an insertion device to a predetermined initial deployment depth.

Accordingly, although an exemplary embodiment of the invention has been shown and described, it is to be understood that all the terms used herein are descriptive rather than limiting, and that many changes, modifications, and substitutions may be made by one having ordinary skill in the art without departing from the spirit and scope of the invention.

### BRIEF DESCRIPTION OF THE SEVERAL VIEWS OF THE DRAWINGS

FIG. 1 depicts a perspective view of an anchor, according to an embodiment;

FIGS. 2A and 2B depict alternative perspective views of the anchor of FIG. 1;

FIGS. 3A through 3E depict various plan views of the anchor of FIG. 1;

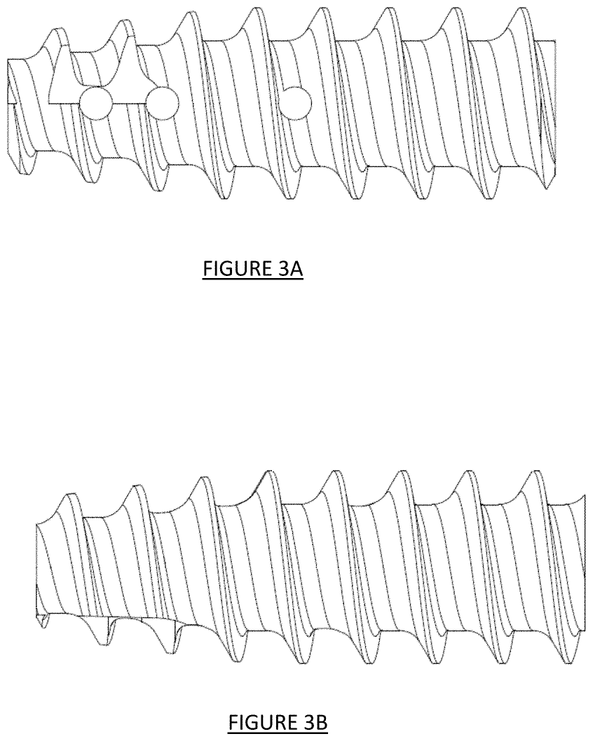

FIGS. 4A through 4D depict various cross-sectional views of the anchor of FIG. 1;

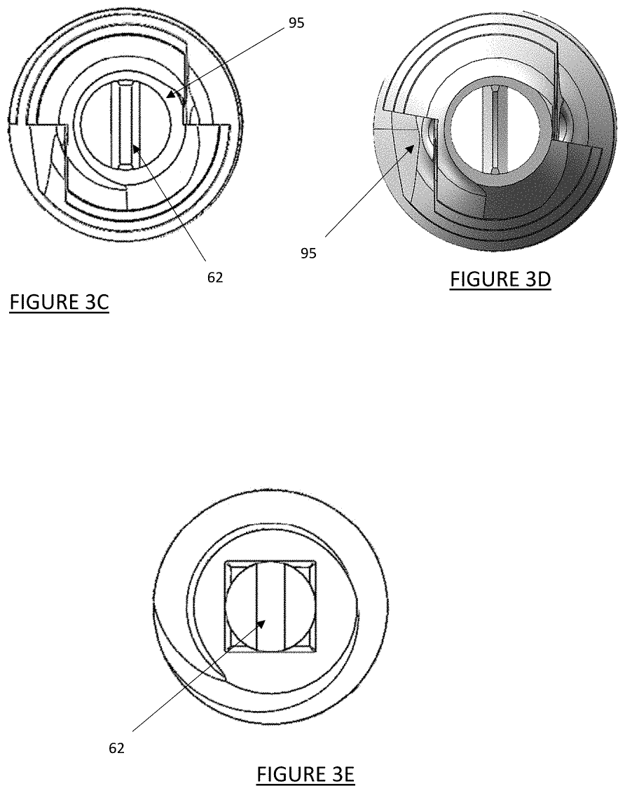

FIG. 5 depicts an enlarged partial view of the cutting flutes of the anchor of FIG. 1;

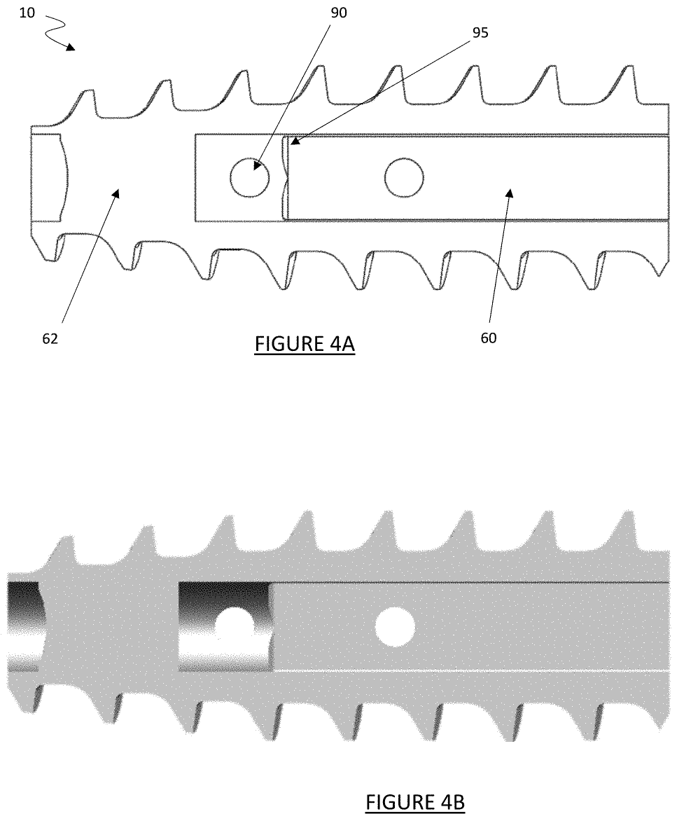

FIG. 6 depicts an exemplary self-packing flowpath for biologic material during anchor rotation and insertion;

FIGS. 7A through 7E depict various plan views of an elongated body component of an insertion and deployment tool for use with an anchor;

FIGS. 8A through 8C depict various plan views of a modular engagement handle component of an insertion and deployment tool for use with an anchor;

FIG. 8D depicts a cross-sectional view of modular engagement handle of FIG. 8C;

FIG. 9A depicts an insertion and/or deployment tool and anchor prior to assembly;

FIG. 9B depicts an assembled insertion and/or deployment tool and anchor;

FIG. 10A depicts a view of a preloaded anchor and insertion and deployment tool with excess suture postponed in a storage card; and

FIG. 10B depicts the preloaded anchor and insertion and deployment tool of FIG. 10A with the card removed.

### DETAILED DESCRIPTION OF THE INVENTION

The disclosures of the various embodiments described herein are provided with sufficient specificity to meet statutory requirements, but these descriptions are not necessarily intended to limit the scope of the claims. The claimed subject matter may be embodied in a wide variety of other ways, may include different steps or elements, and may be used in conjunction with other technologies, including past, present and/or future developments. The descriptions provided herein should not be interpreted as implying any particular order or arrangement among or between various steps or dements except when the order of individual steps or arrangement of elements is explicitly described.

Disclosed herein are a variety of simple-to-insert suture anchors and associated deployment tools which allow for placement of the anchor with little and/or no prior bone preparation, and which facilitates the self-packing and/or venting of bony tissues, blood and/or serum within the anchor interior to promote bony fixation and/or interdigitation between the anchor and surrounding bone structures. In various embodiments, the anchor includes features that allow the use of large suture tapes and/or multiple sutures/suture tapes with a single anchor.

The various systems and devices described herein include the design and manufacturing of suture anchors and related tools having various pre-defined shapes, sizes, widths, spans, thicknesses and/or contours which could be provided in kits of one or more implants of various standard shapes and/or sizes, or could be based, at least partially, off of anatomical shape image information obtained from one or more pre-operative scans (i.e., x-ray, sonogram, CT scan, MRI, etc.) of the patient's anatomy. In such case, the anatomical image information could be identified, scanned, processed and/or analyzed, and a particular implant size, shape, length and/or other features (or a series of implants within a range of sizes, shapes, lengths and/or other features) could be designed and/or selected (i.e., from a library of pre-existing designs and/or from a stockpile of previously manufactured implant components) for use with the patient.

Various embodiments described herein can be used in conjunction with patient specific devices and/or tools that have been constructed specifically for an individual patient, including devices that accommodate the contours and/or dimensions of the patient's bony surfaces and/or other anatomy. The shape of such anatomy can be determined from scans or digital images like a CT Scan or a MRI, and in many cases, such scans can help the surgeon determine an ideal position for cutting and/or preparing anatomical structures for receiving implant components.

If desired, the various suture anchors and/or related tools described herein could be designed and/or manufactured using traditional implant manufacturing techniques, or the various implants and/or components (or portions thereof) could be created using 3D manufacturing techniques (i.e., could be 3D printed using various materials). Such 3D manufacturing techniques could include “just in time” manufacturing in the hospital and/or operating room, if desired, as well as manufacture of multiple copies and/or sizes of suture anchors as needed and/or anticipated.

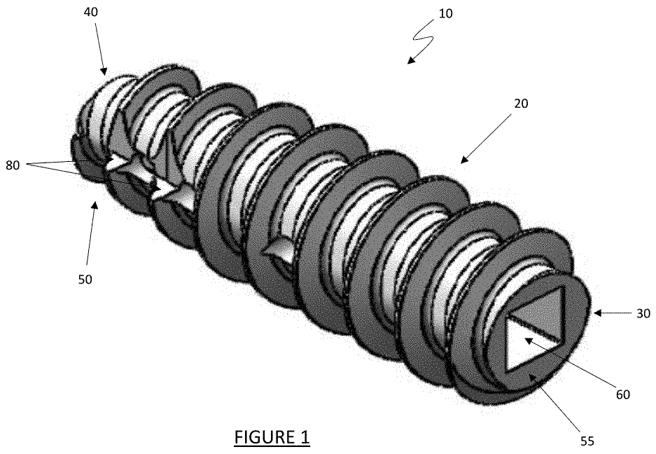

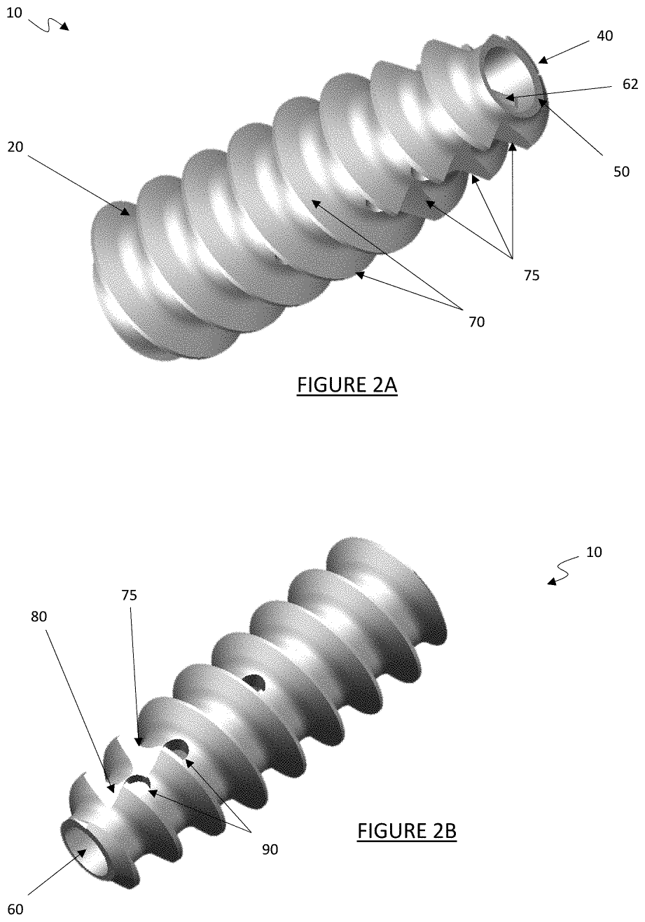

FIG. 1 through 2B depict perspective views of one exemplary embodiment of a suture anchor 10. In this embodiment, the anchor comprises a generally cylindrical body 20 having a proximal end 30 and a distal end 40, the distal end 40 having a somewhat reduced diameter or pointed tip 50, and the proximal end including a relatively flat end section 55. An opening or tunnel 60 extends from the proximal end 30 into the cylindrical body 20, which in various embodiments can extend fully from the proximal end to the distal end of the body or some portion thereof. If desired, a central stem or beam 62 can be provided which extends across, but does not completely close, the tunnel 60, which can provide a variety of advantages to the anchor design, including the provision of attachment locations for suture/suture tape material as well as strengthening, stiffening and/or rigidifying portions of the anchor body and/or anchor tip in a desired manner. A series of one or more externally projecting threads of thread flutes 70 extends outward from the body 20, curving around the body in a helical shape in a well-known manner. In various embodiments, some portion of the thread flutes are desirably continuous around the entire circumference of the threaded portion for much of the length of the body.

In various embodiments, the proximal end 30 of the anchor 10 can include a geometric opening shaped into a square, a triangle a hex or some other shape, which desirable engages with a correspondingly distal end shape of an insertion and deployment tool.

In various embodiments, the distal tip of the anchor can include a relatively flattened or blunt tip 50, which may be cannulated, or the tip may optionally be pointed and/or self-tapping.

In one or more locations along the body, the flutes can include missing, indented, notched and/or depressed sections 75 which can including various curved or relatively flattened surface(s) arranged to form cutting flutes 80, which desirably include edges or cutting surfaces that can abrade and/or cut into the surrounding bony tissues during rotation of the anchor. In the disclosed embodiment, one or more of these depressed portions can be formed proximate to the distal end of the anchor, such that rotation of the anchor brings these cutting surfaces into contact with uncut bony tissues during rotation and/or advancement of the anchor. As depicted, the diameter of the cutting flutes can increase from the distal tip towards the proximal end of the anchor, with portions of the missing or depressed sections 75 and associated cutting surfaces and/or depressions located on reduced diameter portions of the anchor's leading edge (although such missing or depressed sections could alternatively be included on larger diameter portions or other portions of the body, as desired).

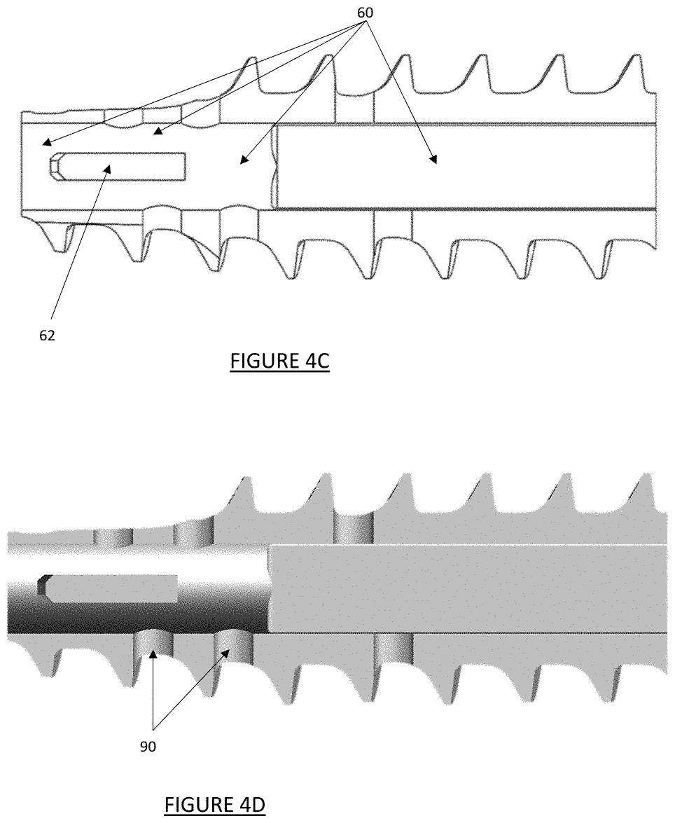

One or more openings or vents 90 can be formed in the body 20 proximate to the cutting flutes 80, with the vents desirably extending inward and desirably in fluid communication with the tunnel 60 of the body 20. During insertion and rotation of the anchor, the cutting flutes 80 present near the distal end of the anchor desirably abrade and/or cut surrounding bony material and also direct biological material (including morselized bone and/or bone chips) into and/or through the vents 90, thereby directing biomaterial into the tunnel 60—thereby packing the tunnel and vents with biomaterials and promoting ingrowth into the anchor. As best seen in FIG. 6, during rotation R of the anchor, the indented or notched portions 75 and the cutting flutes 80 form a recess or scoop which catches bone material (and/or other biologic material) that is passed through the notch, where this material desirably “piles up” against the cutting flute 80 and is pushed or “pressed” into the vents 90 of the anchor (see also FIG. 6).

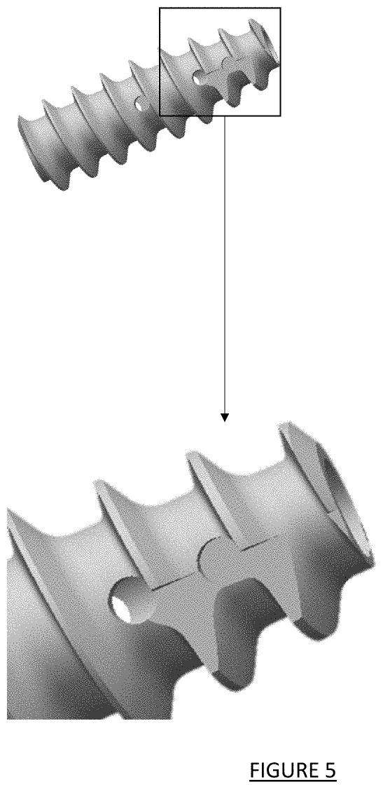

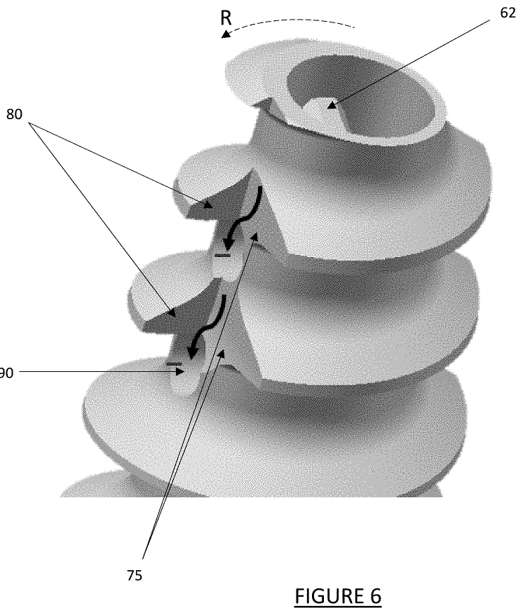

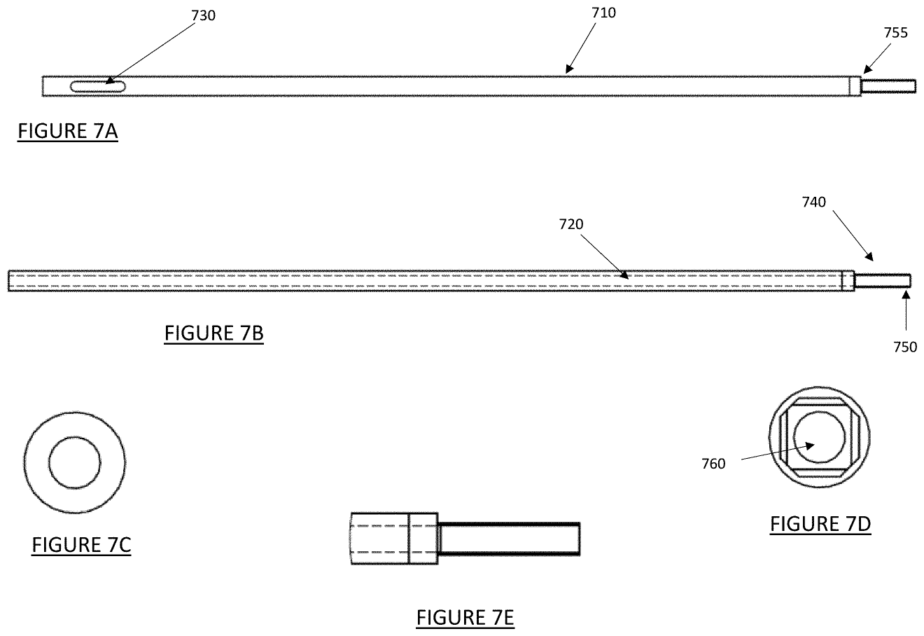

FIGS. 7A through 7E and 8A through 8D depict various components of one exemplary embodiment of an insertion and/or deployment tool 700 which can utilized in conjunction with the anchor embodiments described herein. As best seen in FIG. 7A, the tool can include an elongated body 710, the body desirably incorporating a cannulation or central bore 720 formed therein, with a proximal suture opening 730 in fluid communication with the central 720 bore. A distal tip 740 of the body 710 can include a reduced diameter engagement tip 750 and a distal shoulder region 755, which tip in this embodiment can include a squared outer profile which engages with the opening or tunnel at the proximal end of the anchor, with a portion of the distal tip optionally engaging with a shoulder 95 within the opening in the anchor and/or a portion of the distal shoulder region 755 engaging the relatively flat end section 55 of the anchor 10. The central bore 720 desirably extends through the reduced diameter engagement tip 750, with the distal end of the body desirably including a distal opening 760. FIGS. 8A through 8D depict a modular engagement handle 800 which can be connected to the elongated body, with the handle 800 including a distal tip section 810 which can be secured to the elongated in a variety of ways, including insertion of the distal tip section 810 into a proximal portion of the central bore of the elongated body, and/or by adhering or welding or other securement techniques known in the art.

In at least one exemplary embodiment, the modular engagement handle can comprise an AO disconnect component commonly utilized with surgical instruments. If desired, a ratcheting handle or similar device (not shown) can be engaged with the modular engagement handle and/or AO disconnect to facilitate manipulation and use of the tool by a trained physician. Various similar connection means to an AO connect or Hudson connect may alternatively be utilized with the present invention, such as a “chuck” types well known in the art, that will allow the tool to accommodate a variety of surgical attachments, including ratcheting adapters, torque limiters and/or multipliers, counter torque wrenches, awls, punches, trephines and/or the like.

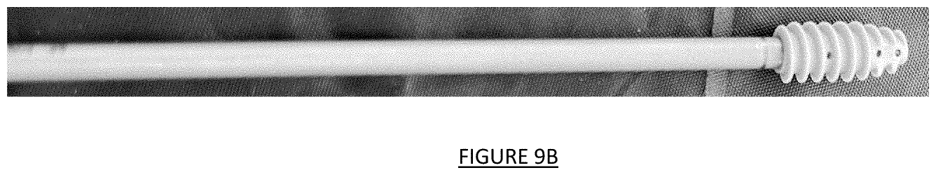

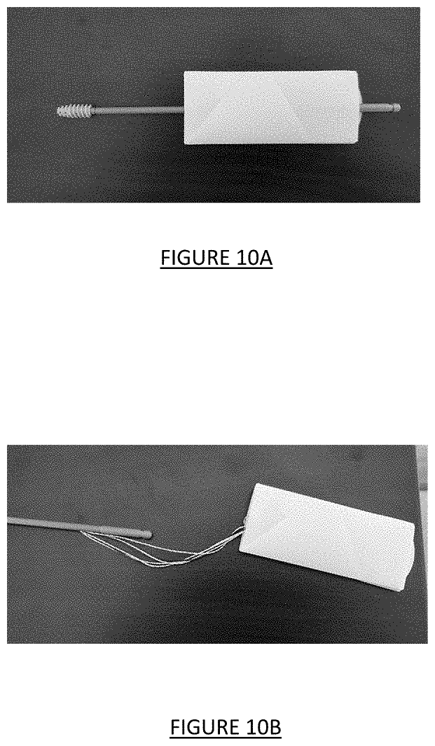

In providing the tool with a modular connection and/or slot at the proximal end, the inserter shaft can easily by reused and/or reprocessed. The shaft can be designed to work with AO connect adaptors with a slot for suture to exit just distal of the AO connection, allowing the shaft and tool to be used with virtually any AO handle, and to allow the shaft to be reprocessed and loaded with another anchor and reused in future cases. Moreover, the slot in the shaft also allows integration with a unique suture card/shaft capability allowing the suture to be removed from the card simply by pulling the shaft free (see FIGS. 10A and 10B).

In use, the proximal opening of an anchor can be slid over and engaged with the reduced diameter engagement tip of the tool, with one or more sutures secured around and/or to the central stem or beam 62 of the anchor, with the suture extending through the opening or tunnel 60 of the anchor, into and through the distal bore and the central bore of the body, and passing through the proximal suture opening of the tool. A projection (not shown) or other component can be utilized to secure the suture, desirably drawing the anchor onto and in secure engagement with the reduced diameter engagement tip at the distal end of the tool. In such an arrangement, the tool can be utilized to insert, advance and secure anchor in a desired position within the bony anatomy (i.e., by rotation of the tool in a desired manner) and then the suture can be loosened from the projection or other securement component with the tool withdrawn from the anchor (while the suture remains attached to the anchor and is drawn through the central bore as the tool is disengaged and withdrawn.

If desired, the sutures and/or suture tapes maybe secured to pins, openings and/or around channels within the periphery of an attached handle. Alternatively, the handle might be omitted and a connection provided on the tool shaft for connection to a power tool, such as a drill. Where an AO connect or Hudson connect are provided, one may appreciate the tool may attach directly to the a handle or to a power tool, via the AO connect or Hudson connect. Other connections between the handle or power and the tool are contemplated.

Desirably, the tool will include a distal tip portion with a transverse cross-section that is generally non-circular and which mates with a corresponding bore of the anchor. For example, where the mating surfaces of the anchor and the tool, whether hexagonal, rectangular, square, elliptical, or otherwise, will allow for a drive mechanism and/or the tool to transfer torque to the anchor for advancing the anchor into the bone. In various embodiments, rotation of the tool and attached anchor can cause rotation of the flutes and advance the anchor into the bony anatomy, and also desirably cause the flutes to cut surrounding biomaterials and “self-pack” some or all of these biomaterials into the tunnel 60 of the anchor, without need for supplemental addition of bone graft or other materials to promote healing and/or bony interdigitation. The cannulation of the anchor and the vent holes desirably allow for blood and biologic healing products to enter the anchor and facilitate healing. Bone may also in grow into the cannulation and vent holes to assist in healing.

In some embodiments, one or more additional vents or openings can be provided into and/or through the anchor body to provide additional passages for bony ingrowth or other objectives, depending upon a variety of design considerations and/or physician preferences. In various embodiments, the cutting flutes present on the distal end of the anchor desirably provide an exit point to material chips. The cutting flutes can include a unique vent hole arrangement placed so as to accept bone chips as the are fed through the cutting flute, along with other biologic material such as blood, serum and/or stem or pluripotent cells.

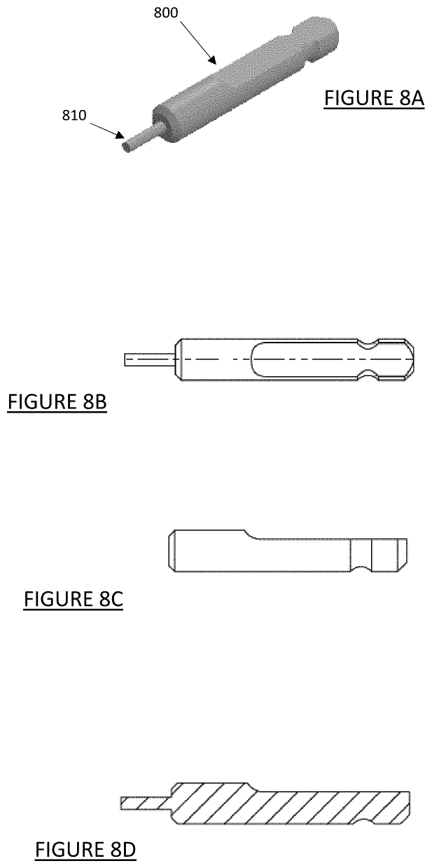

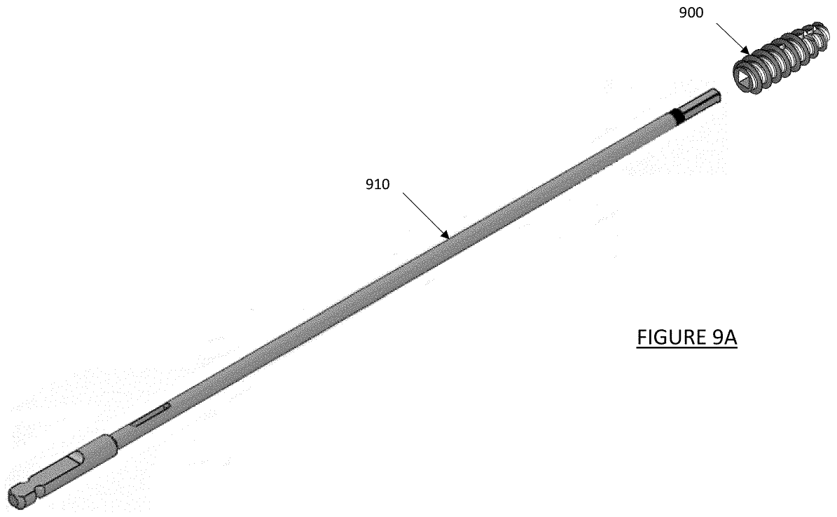

In various embodiments, a bone anchor 900 and associated suture(s) can be provided in a preloaded configuration on an insertion and/or deployment tool 910, such as depicted in FIGS. 9A, 9B, 10A and 10B. In such embodiments, a physician may select a desired tool and associated preloaded anchor from a kit of preloaded tool/anchor combinations, which can greatly simplify preparation and utilization of the anchor during a surgical procedure. If desired, a kit of anchors of different sizes and/or shapes can come preloaded onto reusable driver tools. The driver tools will desirably include slots located at their proximal ends to thereby allow suture to be stored and exit prior to the AO connection (i.e., when the suture storage card is removed prior to use).

If desired, the surgical tool could optionally be reused during the same procedure (such as where an additional anchor and associated sutures may be reloaded onto the tool), or the tool may be recycled and/or resterilized for use by the hospital and/or manufacturer in a subsequent surgical procedure.

The anchor may have a length, in an exemplary embodiment, of approximately 17 mm, although other dimensions are contemplated. In an exemplary embodiment, the anchor may have a pitch of 2.00 mm or a pitch of 1.50 mm. The distance at which the threads begin to taper may be 5.6 mm. The taper angle may be 12 degrees. The width of each thread may be 0.15 mm near the crest. The angle of each flank may be 120 degrees with respect to the root. The angle of the crest with respect to the root may be 93 degrees. The diameter at the root of the anchor may be 4.00 mm or 4.75 mm. The diameter at the crest may be 5.50 mm or 6.00 mm. The diameter at the root may be 4.00 mm when the diameter at the crest is 5.50 mm. The diameter at the root may be 4.75 mm when the diameter at the crest is 6.00 mm. However, it should be appreciated that these dimensions are exemplary, and other dimensions are contemplated.

The disclosed embodiments allow the disclosed anchors to be provided in a preloaded condition, if desired, such that the surgeon has the intraoperative flexibility to quickly and conveniently select a preprepared suture or tape the surgeon feels is best for the surgery. Of course, the disclosed system component also allow for flexibility in the selection of a desired anchor and/or suture/suture tape, which may be assembled and/or modified at any point during the surgical procedure. Because there are many different surgeon preferences for tapes and/or sutures, as well as the potential need for different colors, sizes and/or styles of tape in a single surgery, the disclosed embodiments desirably optionally allow an anchor to be utilized with any style, color and/or size of surgical suture and/or tape, which allows the surgeon to customize their anchor for whatever type of repair he or she needs.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An anchoring system for securing tissue to bone, comprising
an implant comprising a body having an internal cavity, a first lateral vent opening in fluid communication with the internal cavity, and one or more helical cutting flutes positioned on an external surface of the body, the helical cutting flutes including at least one bone harvesting surface which cuts the bone and directs at least a portion of the cut bony material into the first lateral vent and into the internal cavity as the body is rotated into the bone.

2. The anchoring system of claim 1, wherein the first lateral vent opening is positioned between adjacent helical cutting flutes.

3. The anchoring system of claim 1, wherein the one more helical cutting flutes includes at least one notched section positioned proximate to the first lateral vent opening.

4. The anchoring system of claim 3, further comprising a second lateral vent opening in fluid communication with the internal cavity, wherein the second lateral vent opening is longitudinally spaced apart from the first lateral vent opening.

5. The anchoring system of claim 1, further comprising a second lateral vent opening in fluid communication with the internal cavity, wherein the second lateral vent opening is circumferentially spaced apart from the first lateral vent opening.

6. The anchoring system of claim 1, wherein the implant further includes an internal beam extending across the internal cavity with at least a portion of the internal cavity passing along a first side and a second side of the internal beam.

7. The anchoring system of claim 1, wherein the internal beam is positioned proximate to a distal end of the anchor.

8. The anchoring system of claim 1, wherein the internal cavity at a location proximate to a proximal end of the anchor is formed in a non-circular cross-section.

9. The anchoring system of claim 8, further comprising an implantation tool having an elongated shaft with a cannulation therein, a distal end of the elongated shaft having a reduced diameter section with a non-circular cross-section that is substantially similar to the non-circular cross-section of the proximal end of the anchor, wherein the reduced diameter section of the elongated shaft fits within and engages with the internal cavity at the distal end of the anchor.

10. The anchoring system of claim 9, wherein the elongated shaft comprises a lateral opening in a wall of the elongated shaft, the proximal opening in fluid communication with the cannulation.

11. The anchoring system of claim 10, wherein the implantation tool further comprises a modular engagement shaft positioned at a proximal end of the elongated shaft, the modular engagement shaft including a distal tip portion which passes into the cannulation in a proximal end of the elongated shaft, the distal tip portion having a solid cross-section.

12. The anchoring system of claim 1, further comprising a second lateral vent opening in fluid communication with the internal cavity, wherein the second lateral vent opening is longitudinally and circumferentially spaced apart from the first lateral vent opening.

13. The anchoring system of claim 10, wherein the implant further includes an internal beam extending across the internal cavity with at least a portion of the internal cavity passing along a first side and second side of the internal beam,
and at least one suture passing around the external beam and extending through at least a portion of the internal cavity of the anchor,
the at least one suture passing through the canulation of the elongated shaft and extending out of the later opening in the wall of the elongated shaft.

14. The anchoring system of claim 13, wherein the implant further includes an internal beam extending across the internal cavity with at least a portion of the internal cavity passing along a first side and a second side of the internal beam, and the at least one suture passes along both the first and second sides of the internal beam.

15. The anchoring system of claim 9, wherein the implantation tool further comprises a distal shoulder region positioned at a proximal end of the reduced diameter section which engages with and end section of the anchor.

16. The anchoring system of claim 9, wherein the distal end of the elongated shaft includes a transverse surface which at least partially engages with a shoulder positioned within the opening in the anchor.
